(19) United States
(12) Patent Application Publication
(10) Pub. No.: US 2025/0281125 A1
(43) Pub. Date: Sep. 11, 2025

(54) METHOD FOR DETECTING SIGNS OF ATRIAL FIBRILLATION IN NORMAL SINUS RHYTHM AND DEVICE THEREOF

(71) Applicant: CHONNAM NATIONAL UNIVERSITY HOSPITAL, Gwangju (KR)

(72) Inventors: KI HONG LEE, Gwangju (KR); JI WOONG KIM, Gwangju (KR); MYUNG EUN LEE, Gwangju (KR)

(21) Appl. No.: 18/678,085

(22) Filed: May 30, 2024

(30) Foreign Application Priority Data
Mar. 6, 2024 (KR) 10-2024-0032095

(51) Int. Cl. A61B 5/00; A61B 5/353; A61B 5/355; A61B 5/358; A61B 5/361; A61B 5/366; G16H 15/00

(52) U.S. Cl. CPC A61B 5/7275; A61B 5/353; A61B 5/355; A61B 5/358; A61B 5/361; A61B 5/366; A61B 5/7267; G16H 15/00

(57) ABSTRACT

A method for detecting signs of atrial fibrillation in normal sinus rhythm includes acquiring electrocardiogram data of a patient, generating preprocessed electrocardiogram data based on at least some specific regions from the electrocardiogram data, and determining a patient's risk of developing atrial fibrillation using the preprocessed electrocardiogram data and a pretrained atrial fibrillation determination model.

### BACKGROUND

### 1. Field of the Invention

The present disclosure relates to a method for detecting signs of atrial fibrillation in normal sinus rhythm and a device thereof.

### 2. Description of the Related Art

Arrhythmia may be confused with other diseases due to unclear symptoms and many different types thereof. In particular, when atrial fibrillation progresses, it may rapidly lead to serious complications such as heart attack or angina, thus regular diagnosis and caution are required.

Diagnosis of arrhythmia that causes atrial fibrillation is performed through electrocardiogram measurement and determined based on results thereof. In this case, the electrocardiogram measurement is generally carried out in specialized medical facilities, such that it is not easy for patients to measure the electrocardiogram in their daily lives.

In fact, even in patients suspected of having arrhythmia, atrial fibrillation often does not exhibit symptoms when measuring electrocardiogram (ECG), and as a result, patients may be exposed to life-threatening circumstances due to a sudden onset of arrhythmia during their daily lives without any symptom.

Therefore, in order to prevent an occurrence of emergency situation in patients with heart disease, researches on technologies that can measure the electrocardiogram during daily life and technologies that predict an occurrence of arrhythmia or atrial fibrillation using the measured electrocardiogram data are required.

### SUMMARY

It is an object of the present disclosure to provide a method for detecting signs of atrial fibrillation in normal sinus rhythm of a user based on mobile ECG and a device thereof.

Problems to be solved through various embodiments are not limited to the above-described problem, and other problems not described above will be clearly understood by those skilled in the art from the following description.

To achieve the above object, according to an aspect of the present invention, there is provided a method for detecting signs of atrial fibrillation in normal sinus rhythm, including: acquiring electrocardiogram data of a patient; generating preprocessed electrocardiogram data based on at least some specific regions from the electrocardiogram data; and determining a patient's risk of developing atrial fibrillation using the preprocessed electrocardiogram data and a pretrained atrial fibrillation determination model.

Here, the step of generating the preprocessed electrocardiogram data may include removing at least a portion of a first region preset before a specific P-peak and a second region preset after a specific T-peak from the electrocardiogram data.

Here, the step of generating the preprocessed electrocardiogram data may include dividing the preprocessed electrocardiogram data into input data of a first preset time to generate an input data set.

Here, the pretrained atrial fibrillation determination model may be configured to output a probability of the patient's risk of developing atrial fibrillation corresponding to the input of the input data set.

Here, the method for detecting signs of atrial fibrillation in normal sinus rhythm may further include generating an analysis report on the risk of developing atrial fibrillation.

Here, the method for detecting signs of atrial fibrillation in normal sinus rhythm may further include, if the patient's risk of developing atrial fibrillation exceeds a preset reference value, transmitting the analysis report to a preset device of a medical institution.

Here, the pretrained atrial fibrillation determination model may be trained through the steps of: acquiring training electrocardiogram data including first normal sinus rhythm data of patients with a history that atrial fibrillation is developed and second normal sinus rhythm data of patients without the history that atrial fibrillation is developed; labeling the first normal sinus rhythm data and the second normal sinus rhythm data with distinguished marks; and causing the previously generated atrial fibrillation determination model to perform training using the training electrocardiogram data.

Here, the pretrained atrial fibrillation determination model may be configured to determine the patient's risk of developing atrial fibrillation based on at least some of ST segments and QRS complexes of the preprocessed electrocardiogram data.

According to another aspect of the present invention, there is provided a device for detecting signs of atrial fibrillation in normal sinus rhythm, including: an electrocardiogram data processing unit configured to acquire electrocardiogram data of a patient and generate preprocessed electrocardiogram data based on at least some specific regions from the electrocardiogram data; and an atrial fibrillation determination unit configured to determine a patient's risk of developing atrial fibrillation using the preprocessed electrocardiogram data and a pretrained atrial fibrillation determination model.

Here, the electrocardiogram data processing unit may remove at least a portion of a first region preset before a specific P-peak and a second region preset after a specific T-peak from the electrocardiogram data.

Here, the electrocardiogram data processing unit may divide the preprocessed electrocardiogram data into input data of a first preset time to generate an input data set.

Here, the pretrained atrial fibrillation determination model may be configured to output a probability of the patient's risk of developing atrial fibrillation corresponding to the input of the input data set.

Here, the device for detecting signs of atrial fibrillation in normal sinus rhythm may further include a determination result processing unit configured to, if the patient's risk of developing atrial fibrillation exceeds a preset reference value, generate an analysis report on the risk of developing atrial fibrillation.

Here, the determination result processing unit may transmit the analysis report to a preset device of a medical institution.

Here, the device for detecting signs of atrial fibrillation in normal sinus rhythm may further include an atrial fibrillation determination model training unit configured to cause the atrial fibrillation determination model to perform training, wherein the atrial fibrillation determination model training unit performs training of the pretrained atrial fibrillation determination model by: acquiring training electrocardiogram data including first normal sinus rhythm data of patients with a history that atrial fibrillation is developed and second normal sinus rhythm data of patients without the history that atrial fibrillation is developed; labeling the first normal sinus rhythm data and the second normal sinus rhythm data with distinguished marks; and causing the previously generated atrial fibrillation determination model to perform training using the training electrocardiogram data.

Here, the pretrained atrial fibrillation determination model may be configured to determine the patient's risk of developing atrial fibrillation based on at least some of ST segments and QRS complexes of the preprocessed electrocardiogram data.

According to various embodiments, as described above, by providing an environment in which electrocardiogram measurement is possible to enable real-time monitoring during daily life based on mobile ECG using a small number of electrodes, electrocardiogram data may be easily collected.

According to various embodiments, by early detecting signs of atrial fibrillation from electrocardiogram data consisting of normal sinus rhythm measured through mobile ECG, it is possible to provide an environment in which necessary measures can be taken in preparation for an occurrence of an emergency medical situation in patients.

According to various embodiments, by providing a method for early detecting hidden atrial fibrillation signs even in normal sinus rhythm using mobile electrocardiogram and a device thereof, it is possible to contribute to provide an innovative medical environment in which the management of heart disease in patients is improved through cardiac health monitoring and the risk of arrhythmia that may occur in the patients is early diagnosed at the medical sites, etc.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram schematically illustrating the configuration of a device for detecting signs of atrial fibrillation in normal sinus rhythm according to an embodiment;

FIG. 2 is a diagram schematically illustrating an electrocardiogram waveform included in electrocardiogram data acquired by the device according to an embodiment;

FIG. 3 is a flowchart illustrating procedures of an operation for detecting signs of atrial fibrillation in normal sinus rhythm from the electrocardiogram data by the device according to an embodiment;

FIG. 4 is a flowchart illustrating detailed procedures of an operation for detecting signs of atrial fibrillation in normal sinus rhythm from the electrocardiogram data by the device according to an embodiment;

FIG. 5 is a flowchart illustrating procedures of an operation for performing in relation to sign detection of atrial fibrillation in normal sinus rhythm from electrocardiogram data by the device according to an embodiment; and

FIG. 6 is a flowchart illustrating procedures of an operation for causing an atrial fibrillation determination model to perform training in the device according to an embodiment.

### DETAILED DESCRIPTION OF THE INVENTION

The above and other aspects, features, and advantages of the present invention will become apparent from the detailed description of the embodiments to be described in detail below in conjunction with the accompanying drawing. In this regard, it should be understood that the present invention is not limited to the following embodiments and may be embodied in various different ways, and that the embodiments are given to provide complete invention of the present invention and to provide a thorough understanding of the present invention to a person who has a common knowledge in the technical field to which the present invention belongs. The present invention is defined only by the scope of the claims. Hereinafter, the same reference numerals are denoted to the same components.

Although a first, a second, and the like are used to describe various elements, components and/or sections, these elements, components and/or sections are of course not limited by these terms. These terms are merely used to distinguish one element, component and/or section from another element, component and/or section. Therefore, it goes without saying that the first element, first component or first section mentioned below may also be the second element, second component or second section within the technical spirit of the present invention.

Terms used herein are for the purpose of describing particular embodiments only and are not intended to limit the present disclosure thereto. As used herein, singular forms are intended to include the plural forms as well, unless the context clearly indicates otherwise. It will be further understood that the terms “comprises” and/or “comprising,” as used herein, do not preclude the presence or addition of one or more elements other than those mentioned.

Unless otherwise defined, all terms (including technical and scientific terms) used herein have the same meaning as commonly understood by one of ordinary skill in the art to which the present disclosure pertains. Terms, such as those defined in commonly used dictionaries, are not to be construed in an idealized or overly formal sense unless expressly so defined herein.

Hereinafter, various embodiments of the present disclosure will be described with reference to the accompanying drawings. However, the drawings attached to the present specification serve to further understand the technical idea together with the detailed description, such that the present disclosure should not be construed as being limited only to the illustrations of the drawings.

The present disclosure describes a method for detecting signs of atrial fibrillation and a device thereof. To describe in more detail, the present disclosure may describe a method for detecting signs of atrial fibrillation in normal sinus rhythm using electrocardiogram data measured by a mobile device and a device thereof.

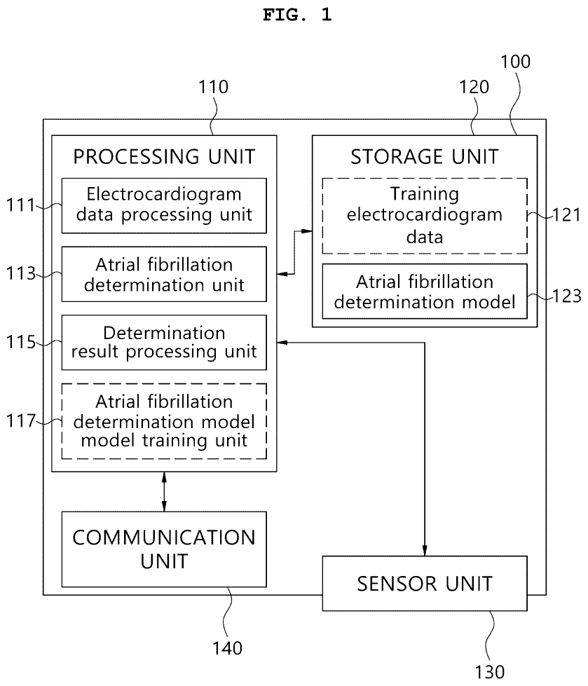

FIG. 1 is a block diagram schematically illustrating the configuration of a device that detects signs of atrial fibrillation in normal sinus rhythm according to an embodiment.

Referring to FIG. 1, a device 100 for detecting signs of atrial fibrillation in normal sinus rhythm (hereinafter, briefly referred to as a device 100) may include a processing unit 110, a storage unit 120, a communication unit 140 and a sensor unit 130.

The processing unit 110 includes at least one processor, and may process various data for an operation of the device 100 through at least one program (application, tool, plug-in, software or the like).

To this end, the processing unit 110 may be classified based on function or scope of the operation. For example, the processing unit 110 may include an electrocardiogram data processing unit 111 configured to acquire and/or preprocess electrocardiogram data, an atrial fibrillation determination unit 113 configured to determine signs of atrial fibrillation from electrocardiogram data using a pretrained atrial fibrillation determination model, and a determination result processing unit 115 configured to process atrial fibrillation determination results.

In addition, the processing unit 110 may further include an atrial fibrillation determination model training unit 117 configured to cause an atrial fibrillation determination model to perform training.

The storage unit 120 may store various data processed by at least one component of the device 100 (e.g., the processing unit 110, a communication unit or the like). The data may include, for example, a program for processing control commands, data processed through the program, or input data and output data related thereto.

To describe in more detail, the storage unit 120 may include an artificial algorithm for control command processing, which includes at least some of an artificial neural network algorithm, a blockchain algorithm, a deep learning algorithm, and a regression analysis algorithm based on at least some of the mechanisms, operators, language models, and big data related thereto.

According to an embodiment, the storage unit 120 may include an atrial fibrillation determination model 123 configured to determine signs of atrial fibrillation from the input electrocardiogram data of a patient.

The atrial fibrillation determination model 123 may include at least some of algorithms of ResNet, recurrent neural network (RNN), long short-term memory (LSTM), and convolutional neural network (CNN).

In addition, the atrial fibrillation determination model 123 may include at least some optimization algorithms of Adagrad, root mean square propagation (RMSprop), adaptive moment estimation (Adam), Adadelta, Nadam, AMSGrad, Nesterov's Accelerated Gradient (NAG) descent, and Nesterov Momentum.

In addition, the storage unit 120 may further include training electrocardiogram data 121 provided for training the atrial fibrillation determination model.

The training electrocardiogram data 121 may include plural electrocardiogram data for a plurality of patients (those with atrial fibrillation experience and/or those without atrial fibrillation). In addition, the training electrocardiogram data 121 may be classified into at least some categories of a training set, a verification set, and a data set for training the atrial fibrillation determination model.

The sensor unit 130 may include at least one electrocardiogram sensor configured to specify electrocardiogram of a patient.

To describe in more detail, the electrocardiogram sensor may include at least one electrode which comes into contact with patient's skin to transmit an electrical signal related to the electrocardiogram. In this case, the sensor unit 130 may include at least some of the functions of a mobile electrocardiogram sensor including one electrode (1-lead).

However, it is not limited thereto, and the sensor unit 130 may include at least some of the functions of a mobile electrocardiogram sensor including a plurality of electrodes.

Referring to FIG. 1, the sensor unit 130 is shown as a component of the device 100, but it is not limited thereto, and may be configured to be attached to and detached from the device 100, or may be configured outside independently from the device 100.

When the sensor unit 130 is configured independently from the device 100, the communication unit 140 may receive electrocardiogram data measured from a specific patient by the sensor unit 130.

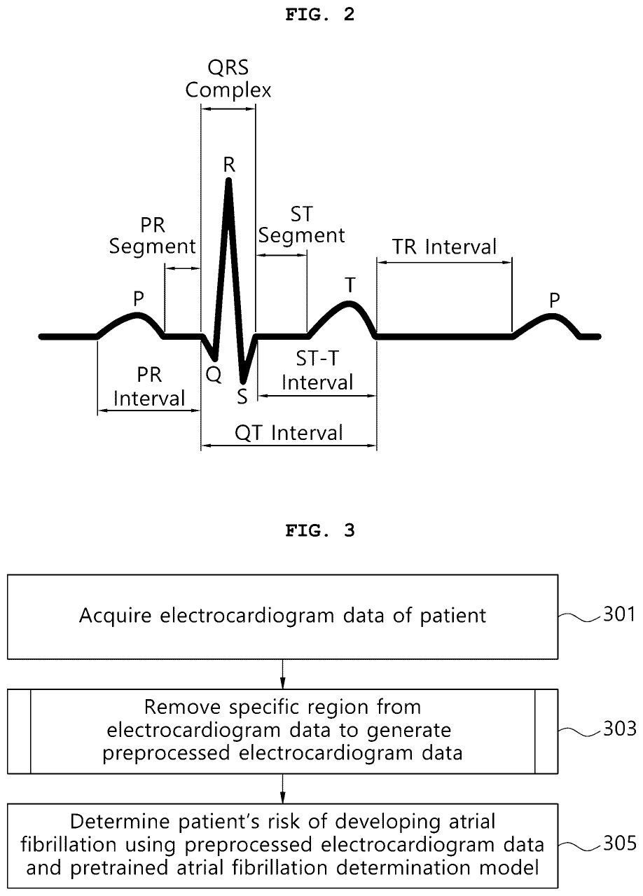

In this regard, FIG. 2 is a diagram schematically illustrating an electrocardiogram waveform included in electrocardiogram data acquired by the device according to an embodiment.

Referring to FIG. 2, the electrocardiogram waveform includes at least some of P waves, QRS complexes and T waves, and based on these, it may be classified into a plurality of intervals and a plurality of segments.

To describe in more detail, the electrocardiogram waveform may be classified into at least some intervals and segments of PR intervals, QT intervals, ST-T intervals, TR intervals, PR segments and ST segments.

In addition, the sensor unit 130 may include at least one sensor for measuring physiological conditions of the patient.

For example, the sensor unit 130 may include one or more sensors of at least one temperature measurement sensor for measuring patient's body temperature, and a pulse measurement sensor for measuring patient's pulse.

According to the above description, it has been described that the sensor unit 130 includes the temperature measurement sensor and/or pulse measurement sensor, but it is not limited thereto, and may be configured to be connected to a temperature measurement sensor and/or pulse measurement sensor provided outside the device 100.

The communication unit 140 may support establishment of a wired communication channel or establishment of a wireless communication channel between inside the device 100 and/or the device 100 and at least one other device (e.g., the user device or a server), and performing communication through the established communication channel.

In addition, although not shown in FIG. 1, the device 100 may further include at least one input/output unit.

The input/output unit may include or be connected to at least some of an input unit (not shown) for inputting data, such as a keyboard, mouse, touch pad, or the like, and an output unit (not shown) for outputting data, such as a display unit (e.g., a display), speaker, driver or the like.

According to various embodiments of the present invention, the device 100 or user device may include at least some of functions of all information and communication devices including a mobile communication terminal, a multimedia terminal, a wired terminal, a fixed terminal, an internet protocol (IP) terminal and the like.

The device 100 is a device for control command processing, and may include at least some functions of a workstation or a large-capacity database, or may be connected thereto through communication.

Hereinafter, a method of acquiring electrocardiogram data measured from a patient, and determining signs of atrial fibrillation in normal sinus rhythm from the electrocardiogram data, which are performed by the device 100, will be described in detail with reference to FIGS. 3 to 6.

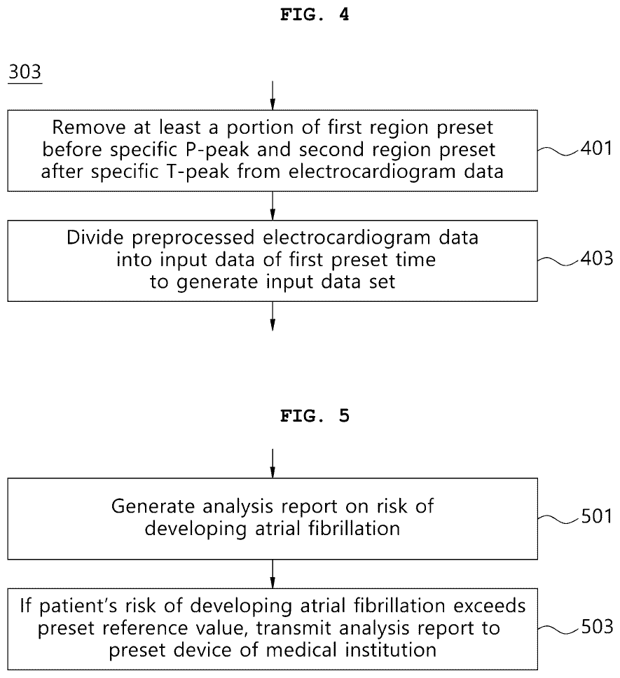

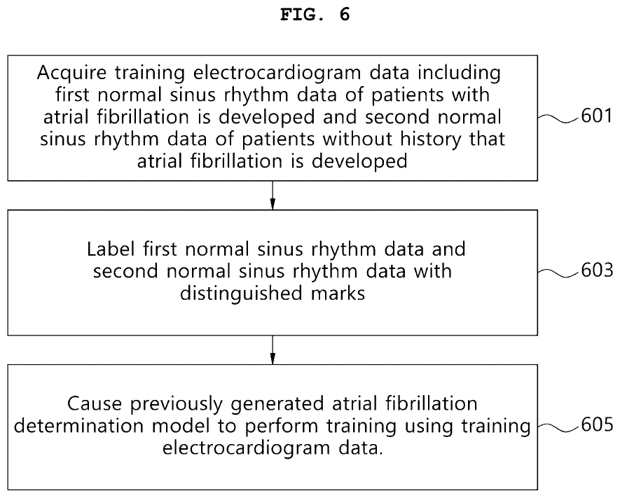

In this regard, FIG. 3 is a flowchart illustrating procedures of an operation for detecting signs of atrial fibrillation in normal sinus rhythm from the electrocardiogram data by the device according to an embodiment, FIG. 4 is a flowchart illustrating detailed procedures of an operation for detecting signs of atrial fibrillation in normal sinus rhythm from the electrocardiogram data by the device according to an embodiment, FIG. 5 is a flowchart illustrating procedures of an operation for performing in relation to sign detection of atrial fibrillation in normal sinus rhythm from electrocardiogram data by the device according to an embodiment, and FIG. 6 is a flowchart illustrating procedures of an operation for causing an atrial fibrillation determination model to perform training in the device according to an embodiment.

First, referring to FIG. 3, in step 301, the electrocardiogram data processing unit 111 may acquire electrocardiogram data of a patient.

To describe it in more detail, when the device 100 includes the sensor unit 130, the electrocardiogram data processing unit 111 may acquire the electrocardiogram data of the patient measured by the sensor unit 130.

According to various embodiments, when the sensor unit 130 is configured independently from the device 100, the electrocardiogram data processing unit 111 may acquire the electrocardiogram data of the patient measured by the sensor unit 130 through the communication unit 140.

In step 303, the electrocardiogram data processing unit 111 may generate preprocessed electrocardiogram data based on at least some specific regions from the electrocardiogram data.

According to an embodiment, as shown in FIG. 4, the electrocardiogram data processing unit 111 may remove at least a portion of a first region preset before a specific P-peak and a second region preset after a specific T-peak from the electrocardiogram data (401), and/or divide the preprocessed electrocardiogram data into input data of a first preset time to generate an input data set (403).

To describe the step in more detail, the 401 electrocardiogram data processing unit 111 may remove a noise region from the electrocardiogram data obtained from the patient to generate the preprocessed electrocardiogram data.

Here, the noise of the electrocardiogram data may include at least some of noise measured together when measuring the electrocardiogram, such as noise generated at the moment of starting or ending the measurement of patient's electrocardiogram by the sensor unit, and noise generated at the moment of attaching and detaching the electrode to the patient.

For example, the electrocardiogram data processing unit 111 may remove a region before a peak of a P wave (P-peak) which is first (initially) identified, and/or a region after a peak of a T wave (T-peak) which is last (finally) identified from the electrocardiogram data acquired from the patient

To describe step 403 in more detail, the electrocardiogram data processing unit 111 may divide the electrocardiogram data from which at least some regions have been removed into preset time units to generate the preprocessed electrocardiogram data.

For example, the electrocardiogram data processing unit 111 may divide the electrocardiogram data into time units preset from 5 to 15 seconds, and more preferably into 10 second units.

However, the electrocardiogram data processing unit 111 is not limited thereto, and may divide the electrocardiogram data to include the predetermined number of specific signals. For example, the electrocardiogram data processing unit 111 may divide the electrocardiogram data so that the divided electrocardiogram data (preprocessed electrocardiogram data) includes the preset number of RR peaks from 10 to 18 peaks, and/or the preset number of RR intervals from 5 to 10 intervals.

Referring to FIG. 4, it is shown that the step 403 is performed after performing the step 401, but it is not limited thereto, and the electrocardiogram data processing unit 111 may perform the step 401 after performing the step 403.

In addition, the electrocardiogram data processing unit 111 may perform only step 401 or only step 403 when performing the operations of the step 303.

Although not shown in FIG. 4, the electrocardiogram data processing unit 111 may perform an operation for removing noise from electrocardiogram data using a filter and/or stabilizing a baseline of the electrocardiogram.

To describe in more detail, the electrocardiogram data processing unit 111 may remove noise from the electrocardiogram data using a filter (e.g., a butterworth filter) which acts on a specific frequency.

Here, the butterworth filter may be configured to act on at least some frequencies from 0 to 1 Hz, for example, 0.5 Hz. However, it is not limited thereto, and the frequency on which the butterworth filter acts or operates may be changed depending on settings.

In addition, the electrocardiogram data processing unit 111 may stabilize the baseline of the electrocardiogram data by estimating the baseline based on a moving average in the electrocardiogram data, and subtracting the estimated baseline from the electrocardiogram data (or the electrocardiogram waveform of the electrocardiogram data).

Further, the electrocardiogram data processing unit 111 may scale the electrocardiogram data so that input values for the atrial fibrillation determination model 123 are uniformly distributed. For example, the electrocardiogram data processing unit 111 may scale the electrocardiogram data so as to be a value within a preset range (e.g., −1 to 1). However, it is not limited thereto, and the scaling range of the electrocardiogram data may be changed depending on settings.

In step 305, the atrial fibrillation determination unit 113 may determine a patient's risk of developing atrial fibrillation using the preprocessed electrocardiogram data and the pretrained atrial fibrillation determination model.

To describe in more detail, the atrial fibrillation determination unit 113 may input the preprocessed electrocardiogram data into the pretrained atrial fibrillation determination model 123 and obtain an output of the atrial fibrillation determination model 123.

Here, the atrial fibrillation determination unit 113 may obtain the output of the atrial fibrillation determination model 123 corresponding to the input preprocessed electrocardiogram data as the patient's risk of developing atrial fibrillation.

According to an embodiment, the atrial fibrillation determination model 123 may be configured to output the risk of developing atrial fibrillation as a probability value (%) when plural preprocessed electrocardiogram data consisting of first time units are input.

However, it is not limited thereto, and the atrial fibrillation determination model 123 may be configured to output the risk of developing atrial fibrillation in various types of values, such as outputting the risk of developing atrial fibrillation as a decimal value having a specific value (e.g., 1) as the maximum value.

The atrial fibrillation determination unit 113 may end the embodiment shown in FIG. 3 when performing step 305.

In addition, although not disclosed through FIGS. 3 and 4, as shown in FIG. 5, the atrial fibrillation determination unit 113 may generate an analysis report on the risk of developing atrial fibrillation output from the atrial fibrillation determination model 123 (501).

To describe in more detail, the atrial fibrillation determination unit 113 may generate an analysis report including at least some pieces of information of the risk of developing atrial fibrillation, atrial fibrillation history information, and personal information of the patient.

To this end, the storage unit 120 may store at least some pieces of information of the atrial fibrillation history information, and personal information of the patient, or may receive at least some pieces of information of the atrial fibrillation history information, and personal information of the patient from a preset server.

Here, the atrial fibrillation history information may include at least some pieces of information related to the patient's atrial fibrillation, such as the presence or absence of an atrial fibrillation history, a point of time (e.g., year, month, day, etc.) when the atrial fibrillation is developed in the case of having atrial fibrillation history, a treatment hospital and the like. To describe in more detail, the atrial fibrillation history information may include information on the most recent point in time when the atrial fibrillation is developed.

Here, the personal information may include at least some pieces of information of various information related to the patient, such as a name, age, date of birth, address, height, weight of the patient, and contact address of a guardian device.

Thereafter, if the patient's risk of developing atrial fibrillation exceeds a preset reference value as a result of the output from the atrial fibrillation determination model 123, the atrial fibrillation determination unit 113 may transmit the analysis report to a preset device of a medical institution (503).

Here, the preset device of the medical institution may include a preset device of a medical hospital for the patient. However, it is not limited thereto, and the atrial fibrillation determination unit 113 may transmit the generated analysis report to at least some of various preset devices, such as a guardian device and devices related to emergency rescue services.

Here, the emergency rescue service may include at least some of the services capable of emergency communication, such as emergency medical and police services.

According to an embodiment, the preset reference value in relation to the patient's risk of developing atrial fibrillation may be set to one value, or may be set to two or more different values.

For example, when the preset reference value is set to one value, if the patient's risk of developing atrial fibrillation exceeds the reference value as a result of the output from the atrial fibrillation determination model 123, the atrial fibrillation determination unit 113 may transmit the analysis report to a first device (or a first device list) (e.g., the guardian device and the device of the emergency rescue service) which is preset to include at least one of the preset device of the medical institution, the guardian device, and the device related to the emergency rescue service.

For another example, there may be a case where the preset value is set as a first reference value and a second reference value higher than the first reference value.

In this case, if the patient's risk of developing atrial fibrillation exceeds the first reference value and is the second reference value or less, the atrial fibrillation determination unit 113 may transmit the analysis report to a second device (or a second device list) (e.g., the guardian device) which is preset to include at least one of the preset device of medical institution, the guardian device, and the devices related to emergency rescue services.

On the other hand, if the patient's risk of developing atrial fibrillation exceeds the second reference value, the atrial fibrillation determination unit 113 may transmit the analysis report to a preset third device (or third device list) (e.g., the guardian device, the device of the emergency medical service, and police device) which is preset to include at least one of the preset device of the medical institution, the guardian device, and the devices related to emergency rescue services.

At least some (501 and/or 503) of the operations (501 to 503) described through FIG. 5 may be processed after performing the step 305 shown in FIG. 3. In addition, at least some of the operations described through FIG. 5 may be performed based on a request from a user (e.g., a patient and/or a designated medical professional).

The atrial fibrillation determination model 123, which outputs the patient's risk of developing atrial fibrillation corresponding to the input of the preprocessed electrocardiogram data as described above, may be trained as shown in FIG. 6.

The operations shown in FIG. 6 are processed by the electrocardiogram data processing unit 111 and/or the atrial fibrillation determination model training unit 117 (hereinafter, referred to as a training unit 117), and may be described that the operations shown in FIG. 6 will be processed by the training unit 117 in the following description.

In step 601, the training unit 117 may acquire training electrocardiogram data including first normal sinus rhythm data (first electrocardiogram data) of patients with a history (past history) that atrial fibrillation is developed and second normal sinus rhythm data (second electrocardiogram data) of patients without the history that atrial fibrillation is developed.

In step 603, the training unit 117 may label at least some of the training electrocardiogram data with specific marks. For example, the training unit 117 may label the first normal sinus rhythm data and the second normal sinus rhythm data with distinguished marks.

To describe in more detail, the training unit 117 may set a label value of 1 for the first normal sinus rhythm data and a label value of 0 for the second normal sinus rhythm data.

However, it is not limited thereto, and the label value for the first normal sinus rhythm data and/or the label value for the second normal sinus rhythm data may be changed according to settings.

In addition, the training unit 117 may generate preprocessed training electrocardiogram data by performing at least some operations of noise removal, baseline stabilization, and scaling on at least some of the training electrocardiogram data.

Here, preprocessing of the training electrocardiogram data may be performed based on at least some operations of the preprocessing operations performed in the step 303.

In step 605, the training unit 117 may cause the previously generated atrial fibrillation determination model to perform training using the training electrocardiogram data. To describe in more detail, the training unit 117 may cause the previously generated atrial fibrillation determination model to perform training using the labeled and/or preprocessed training electrocardiogram data.

Accordingly, the atrial fibrillation determination model 123 may be a result of (or a model generated according to) performing training of the previously generated atrial fibrillation determination model.

To describe in more detail, the previously generated atrial fibrillation determination model may be an artificial intelligence model generated so as to output the risk of developing atrial fibrillation based on the input electrocardiogram data (and/or the preprocessed electrocardiogram data). Based on this, the atrial fibrillation determination model 123 may be a result of performing training of the previously generated atrial fibrillation determination model based on at least some of the operations shown in FIG. 6.

The previously generated atrial fibrillation determination model may be configured to determine the risk of developing atrial fibrillation based on at least some of the specific segments and complexes of the electrocardiogram data (and/or the preprocessed electrocardiogram data), or assign weights to determine the risk of developing atrial fibrillation during the learning process.

For example, the atrial fibrillation determination model may determine the patient's risk of developing atrial fibrillation based on at least some values (and/or changes in the values) of ST segments and QRS complexes of the electrocardiogram data.

In addition, the atrial fibrillation determination model may be configured to assign weights to determine the patient's risk of developing atrial fibrillation based on at least some values (and/or changes in the values) of ST segments and QRS complexes of the electrocardiogram data.

As described above, the atrial fibrillation determination model is trained using the electrocardiogram data (or the preprocessed electrocardiogram data) consisting of normal sinus rhythm as the training electrocardiogram data, and may be trained to determine the risk of developing atrial fibrillation from electrocardiogram data having normal sinus rhythm.

Based on this, the atrial fibrillation determination model 123 may further determine the risk of developing atrial fibrillation based on at least some values (and/or changes in the values) of ST segments and QRS complexes of the electrocardiogram data, or assign weights to determine the risk of developing atrial fibrillation.

According to various embodiments, as described above, by providing an environment in which electrocardiogram measurement is possible to enable real-time monitoring during daily life of electrodes, based on mobile ECG using a small number electrocardiogram data may be easily collected.

According to various embodiments, by early detecting signs of atrial fibrillation from electrocardiogram data consisting of normal sinus rhythm measured through mobile ECG, it is possible to provide an environment in which necessary measures can be taken in preparation for an occurrence of an emergency medical situation in patients.

According to various embodiments, by providing a method for early detecting hidden atrial fibrillation signs even in normal sinus rhythm using mobile electrocardiogram and a device thereof, it is possible to contribute to provide an innovative medical environment in which the management of heart disease in patients is improved through cardiac health monitoring and the risk of arrhythmia that may occur in the patients is early diagnosed at the medical sites, etc.

As described above, although the embodiments have been described with reference to the limited drawings, it will be apparent to those skilled in the art that various modifications and alternations may be applied thereto based on the various embodiments.

For example, adequate effects may be achieved even if the foregoing processes and methods are carried out in different order than those described above, and/or the above-described elements, such as systems, structures, devices, or circuits, are combined or coupled in different forms and modes than those described above, or substituted or switched with other components or equivalents.

In particular, when describing with reference to the flowchart, it has been described that a plurality of steps are configured and the steps are sequentially executed in a designated order, but it is not necessarily limited to the designated order.

In other words, executing by changing or deleting at least some of the steps described in the flowchart or adding at least one step is applicable as an embodiment, and executing one or more steps in parallel may also be applicable as an embodiment. That is, it is not limited to that the steps are necessarily operated in a time-series order, and should be included in various embodiments of the present disclosure.

Therefore, other implements, other embodiments, and equivalents to claims are within the scope of claims to be described below.

What is claimed is:

1. A method for detecting signs of atrial fibrillation in normal sinus rhythm, the method comprising:
acquiring electrocardiogram data of a patient;
generating preprocessed electrocardiogram data based on at least some specific regions from the electrocardiogram data; and
determining a patient's risk of developing atrial fibrillation using the preprocessed electrocardiogram data and a pretrained atrial fibrillation determination model.

2. The method according to claim 1, wherein the generating of the preprocessed electrocardiogram data comprises removing at least a portion of a first region preset before a specific P-peak and a second region preset after a specific T-peak from the electrocardiogram data.

3. The method according to claim 1, wherein the generating of the preprocessed electrocardiogram data comprises dividing the preprocessed electrocardiogram data into input data of a first preset time to generate an input data set.

4. The method according to claim 3, wherein the pretrained atrial fibrillation determination model is configured to output a probability of the patient's risk of developing atrial fibrillation corresponding to the input of the input data set.

5. The method according to claim 1, further comprising generating an analysis report on the risk of developing atrial fibrillation.

6. The method according to claim 5, further comprising, if the patient's risk of developing atrial fibrillation exceeds a preset reference value, transmitting the analysis report to a preset device of a medical institution.

7. The method according to claim 1, wherein the pretrained atrial fibrillation determination model is trained through a process comprising:
acquiring training electrocardiogram data including first normal sinus rhythm data of patients with a history that atrial fibrillation is developed and second normal sinus rhythm data of patients without the history that atrial fibrillation is developed;
labeling the first normal sinus rhythm data and the second normal sinus rhythm data with distinguished marks; and
causing the previously generated atrial fibrillation determination model to perform training the training electrocardiogram data.

8. The method according to claim 1, wherein the pretrained atrial fibrillation determination model is configured to determine the patient's risk of developing atrial fibrillation based on at least some of ST segments and QRS complexes of the preprocessed electrocardiogram data.

9. A device for detecting signs of atrial fibrillation in normal sinus rhythm, the device comprising:
an electrocardiogram data processing unit configured to acquire electrocardiogram data of a patient and generate preprocessed electrocardiogram data based on at least some specific regions from the electrocardiogram data; and
an atrial fibrillation determination unit configured to determine a patient's risk of developing atrial fibrillation using the preprocessed electrocardiogram data and a pretrained atrial fibrillation determination model.

10. The device according to claim 9, wherein the electrocardiogram data processing unit removes at least a portion of a first region preset before a specific P-peak and a second region preset after a specific T-peak from the electrocardiogram data.

11. The device according to claim 9, wherein the electrocardiogram data processing unit divides the preprocessed electrocardiogram data into input data of a first preset time to generate an input data set.

12. The device according to claim 11, wherein the pretrained atrial fibrillation determination model is configured to output a probability of the patient's risk of developing atrial fibrillation corresponding to the input of the input data set.

13. The device according to claim 9, further comprising a determination result processing unit configured to, if the patient's risk of developing atrial fibrillation exceeds a preset reference value, generate an analysis report on the risk of developing atrial fibrillation.

14. The device according to claim 13, wherein the determination result processing unit transmits the analysis report to a preset device of a medical institution.

15. The device according to claim 9, further comprising an atrial fibrillation determination model training unit configured to cause the atrial fibrillation determination model to perform training,
wherein the atrial fibrillation determination model training unit performs training of the pretrained atrial fibrillation determination model by:
acquiring training electrocardiogram data including first normal sinus rhythm data of patients with a history that atrial fibrillation is developed and second normal sinus rhythm data of that atrial fibrillation is patients without the history developed;
labeling the first normal sinus rhythm data and the second normal sinus rhythm data with distinguished marks; and
causing the previously generated atrial fibrillation determination model to perform training using the training electrocardiogram data.

16. The device according to claim 9, wherein the pretrained atrial fibrillation determination model is configured to determine the patient's risk of developing atrial fibrillation based on at least some of ST segments and QRS complexes of the preprocessed electrocardiogram data.
